(19) United States
(12) Patent Application Publication
(10) Pub. No.: US 2026/0102493 A1
(43) Pub. Date: Apr. 16, 2026

(54) ALLOGENEIC DENDRITIC CELL (alloDC) TUMOR VACCINE, AND PREPARATION METHOD AND USE THEREOF

(71) Applicants: ZHONGSHAN FRONTIERGATE BIOPHARM CO., LTD, Zhongshan (CN); SHENZHEN FRONTIERGATE BIOTECHNOLOGY CO., LTD., Shenzhen (CN)

(72) Inventor: Yang XU, Guangzhou (CN)

(21) Appl. No.: 19/029,764

(22) Filed: Jan. 17, 2025

Related U.S. Application Data
(63) Continuation of application No. PCT/CN2024/121825, filed on Sep. 27, 2024.

(30) Foreign Application Priority Data
Oct. 27, 2023 (CN) 202311407954.X

(51) Int. Cl. A61K 40/19; A61K 40/31; A61K 40/42; A61K 40/50; A61P 37/04; C07K 14/725; C07K 16/40; C12N 15/86

(52) U.S. Cl. CPC A61K 40/19; A61K 40/31; A61K 40/4253; A61K 40/50; A61P 37/04; C07K 14/7051; C07K 16/40; C12N 15/86; C12Y 306/05002; C12N 2740/15043

(57) ABSTRACT

An allogeneic dendritic cell (alloDC) tumor vaccine, and a preparation method and use thereof are provided, belonging to the technical field of tumor cell vaccines. An allogeneic chimeric antigen receptor (CAR) dendritic cell (DC) tumor vaccine is an alloDC that recombinantly expresses a CAR and a tumor vaccine. The alloDC serves as a basic cell, where an allogeneic cell for cell therapy shows characteristics of expanding cell sources, improving cell quality, and shortening cell preparation time. Meanwhile, effectiveness and safety of the allogeneic CAR-DC tumor vaccine are verified for tumor treatment. The development of such a novel vaccine overcomes a long-standing limitation of the cell therapy relying on autologous cells, and is conducive to expanding therapeutic application and scale of the CAR-DC and improving a quality of cell preparation, thereby providing new strategy and application for conquering solid tumors.

### CROSS REFERENCE TO RELATED APPLICATION

The present application is a national stage application of International Patent Application No. PCT/CN2024/121825, filed on Sep. 27, 2024, which claims priority to Chinese Patent Application No. 202311407954.X filed to the China National Intellectual Property Administration (CNIPA) on Oct. 27, 2023 and entitled “ALLOGENEIC DENDRITIC CELL (alloDC) TUMOR VACCINE, AND PREPARATION METHOD AND USE THEREOF”, which is incorporated herein by reference in its entirety.

### REFERENCE TO SEQUENCE LISTING

A computer readable XML file entitled “GWPCTP20241006899”, that was created on Dec. 19, 2024, with a file size of about 12, 077 bytes, contains the sequence listing for this application, has been filed with this application, and is hereby incorporated by reference in its entirety.

### TECHNICAL FIELD

The present disclosure belongs to the technical field of tumor cell vaccines, and specifically relates to an allogeneic dendritic cell (alloDC) tumor vaccine, and a preparation method and use thereof.

### BACKGROUND

Tumor immunotherapy has entered an era of diversification, among which immunological technologies based on cell therapy have made remarkable progress in recent years. The cells used for cell therapy include chimeric antigen receptor-T cells (CAR-T), T cell receptor-T cells (TCR-T), dendritic cells (DC) loaded with tumor antigens, natural killer cells (NK), macrophages, etc. The core idea of these cell therapies is to obtain corresponding immune cells from the patient's body, transform, cultivate, and amplify them to make them into engineered cells with certain anti-tumor functions, and then re-infuse them to the body to induce strong anti-tumor immune response. Cell therapy is still a rapidly developing field and a promising technical means to conquer tumors.

T cell therapies such as CAR-T have achieved great success in treating leukemia, but such therapies have failed in the vast majority of solid tumors. On the one hand, this reflects the tolerance of the immune microenvironment of solid tumors, and on the other hand, it also confirms the current situation of poor efficacy of single T cell therapy.

In previous studies, it was found that engineered DC cells (CAR-DC) could effectively reverse the immune microenvironment of tumors (patent CN112830974A). In addition, CAR-DC-loaded tumor antigens (CAR-DC vaccine) could also effectively inhibit tumor growth (patent CN114457117A). Accordingly, the potential of CAR-DC vaccine had been confirmed in anti-tumor effect.

However, the cell source mainly challenges scientists in most cell therapies. This is especially true for DCs. Studies have shown that DCs are less abundant in vivo, and their purification technology also lacks a desirable marker. As a result, in vitro DC preparation methods generally adopt directed differentiation of monocytes to obtain the DCs. A limited number of DCs obtained in this way may become a limitation of DC cell immunotherapy.

### SUMMARY

In view of this, the object of the present disclosure is to provide an alloDC tumor vaccine, which can not only overcome the limitations of DC-based immunotherapy, but also show desirable tumor treatment effect and safety, thereby confirming the feasibility of allogeneic DC cell therapy.

The present disclosure provides an allogeneic CAR-DC tumor vaccine, where the allogeneic CAR-DC tumor vaccine is an alloDC that recombinantly expresses a CAR and a tumor vaccine.

Preferably, the alloDC is a cell therapy-targeted DC derived from different individuals of a same species.

Preferably, the CAR has an amino acid sequence shown in SEQ ID NO: 6.

Preferably, a source gene of the tumor vaccine is at least one selected from the group consisting of TP53, PIK3CA, LRP1B, KRAS, APC, FAT4, KMT2D, KMT2C, BRAF, ARID1A, FAT1, PTEN, ATM, ZFHX3, CREBBP, GRIN2A, NF1, PDE4DIP, PTPRT, TRRAP, RNF213, PREX2, SPEN, ERBB4, KMT2A, RB1, BRCA2, FBXW7, CARD11, PTPRB, ROS1, ATRX, UBR5, NOTCH1, POLQ, EP300, NCOR1, SETD2, SMARCA4, CHD4, MTOR, NCOR2, MED12, RANBP2, ARID2, ZNF521, SETBP1, POLE, CTNNB1, EGFR, P53, and IDH.

Preferably, the tumor vaccine is at least one selected from the group consisting of a P53 R273H mutant peptide, a KRAS G12V mutant peptide, and a KRAS G12C mutant peptide.

Preferably, the p53 R273H mutant peptide has an amino acid sequence shown in SEQ ID NO: 7.

Preferably, the KRAS G12V mutant peptide has an amino acid sequence shown in SEQ ID NO: 8.

The present disclosure further provides a preparation method of the allogeneic CAR-DC tumor vaccine, including the following steps:
constructing a recombinant lentiviral vector containing gene sequences encoding the CAR and the tumor vaccine;
conducting lentiviral packaging using the recombinant lentiviral vector to obtain a recombinant lentivirus; and
infecting the alloDC or a precursor cell or a progenitor cell thereof with the recombinant lentivirus to obtain or culture the allogeneic CAR-DC tumor vaccine.

Preferably, the alloDC or the precursor cell or the progenitor cell thereof is derived from at least one selected from the group consisting of a peripheral blood cell (PBC), a bone marrow cell (BMC), a hematopoietic stem cell (HSC), an embryonic stem cell (ESC), and an induced pluripotent stem cell (iPSC).

Preferably, the recombinant lentiviral vector includes an expression element for expressing the gene sequences encoding the CAR and the tumor vaccine; and
the expression element for expressing the gene sequences encoding the CAR and the tumor vaccine includes a promoter, a CAR-encoding gene, an expression regulation element, and a tumor vaccine-encoding gene.

Preferably, the promoter includes elongation factor 1 alpha (EF1A); and the EF1A has a nucleotide sequence shown in SEQ ID NO: 4.

Preferably, the CAR-encoding gene has a nucleotide sequence shown in SEQ ID NO: 1.

Preferably, the tumor vaccine-encoding gene has a nucleotide sequence shown in SEQ ID NO: 2 or SEQ ID NO: 3.

Preferably, the expression regulation element includes internal ribosome entry site (IRES); and the IRES has a nucleotide sequence shown in SEQ ID NO: 5.

The present disclosure further provides an anti-tumor pharmaceutical composition, including the allogeneic CAR-DC tumor vaccine or an allogeneic CAR-DC tumor vaccine prepared by the preparation method and a medically acceptable auxiliary material.

The present disclosure further provides use of the allogeneic CAR-DC tumor vaccine or an allogeneic CAR-DC tumor vaccine prepared by the preparation method in preparation of a drug for treating a cancer in a subject in need thereof.

Preferably, the cancer is a solid cancer being any one selected from the group consisting of breast cancer, colorectal cancer, pancreatic cancer, liver cancer, lung cancer, and ovarian cancer.

The present disclosure provides an allogeneic CAR-DC tumor vaccine, where the allogeneic CAR-DC tumor vaccine is an alloDC that recombinantly expresses a CAR and a tumor vaccine. The alloDC serves as a basic cell, where an allogeneic cell for cell therapy shows characteristics of expanding cell sources, improving cell quality, and shortening cell preparation time. Meanwhile, effectiveness and safety of the allogeneic CAR-DC tumor vaccine are verified for tumor treatment. Experimental results indicate that development of such a novel vaccine overcomes a long-standing limitation of the cell therapy relying on autologous cells, and is conducive to expanding therapeutic application and scale of the CAR-DC and improving a quality of cell preparation, thereby providing new strategy and application for conquering solid tumors.

### BRIEF DESCRIPTION OF THE DRAWINGS

FIG. 1 shows the test results of humanized mouse modeling;

FIG. 2 shows a schematic diagram of the CAR-DC in the recombinant lentiviral vector;

FIG. 3 shows a schematic diagram of elements of a CAR-DC tumor vaccine in the recombinant lentiviral vector;

FIG. 4 shows expression of CAR protein and CD11c on cell surface of autologous human CAR-DC and CAR-DC vaccine derived from humanized mice;

FIG. 5 shows expression of CAR protein and CD11c on cell surface of the allogeneic CAR-DC vaccine derived from peripheral blood mononuclear cells (PBMCs);

FIG. 6 shows tumor growth curves of each group of mice treated in vivo (CAR-DC-Kras^{G12V}); and

FIG. 7 shows tumor growth curves of each group of mice treated in vivo (CAR-DC-P53^{R273H})

### DETAILED DESCRIPTION OF THE EMBODIMENTS

The present disclosure provides an allogeneic CAR-DC tumor vaccine, wherein the allogeneic CAR-DC tumor vaccine is an alloDC that recombinantly expresses a CAR and a tumor vaccine.

In the present disclosure, the alloDC is preferably a cell therapy-targeted DC derived from different individuals of a same species. The alloDC injected into a patient to be treated not only does not cause rejection reaction of the body, but also can achieve effective anti-tumor and cancer inhibition effects. The alloDC shows desirable therapeutic universality, such that therapeutic drugs can be prepared in advance for the patient population, ensuring the timeliness of cancer treatment.

In the present disclosure, the CAR preferably has an amino acid sequence shown in SEQ ID NO: 6 (MALPVTALLLPLALLLHAARPQVQLLESGGGLVQPGGSLRLSCAASGFTFSSYTMSWVRQA PGQALEWMGTISSRGTYTYYPDSVKGRFTISRDNAKNSLYLQMNSLRAEDTAVYYCAREAIF THWGRGTLVTVSSGGGGSGGGGSGGGGSDIQLTQSPSSLSASVGDRVTITCKASQDINNYHS WYQQKPGQAPRLLIYRANRLVDGVPDRFSGSGYGTDFTLTINNIESEDAAYYFCLKYNVFPY TFGQGTKVEIKTTTPAPRPPTPAPTIASQPLSLRPEACRPAAGGAVHTRGLDFACDIYIWAPLA GTCGVLLLSLVITLYCRWPPSAACSGKESVVAIRTNSQSDFHLQTYGDEDLNELDPHYEMRL KIQVRKAAITSYEKSDGVYTGLSTRNQETYETLKHEKPPQ).

In the present disclosure, a source gene of the tumor vaccine is preferably at least one selected from the group consisting of TP53, PIK3CA, LRP1B, KRAS, APC, FAT4, KMT2D), KMT2C, BRAF, ARID1A, FAT1, PTEN, ATM, ZFHX3, CREBBP, GRIN2A, NF1, PDE4DIP, PTPRT, TRRAP, RNF213, PREX2, SPEN, ERBB4, KMT2A, RB1, BRCA2, FBXW7, CARD11, PTPRB, ROS1, ATRX, UBR5, NOTCH1, POLQ, EP300, NCOR1, SETD2, SMARCA4, CHD4, MTOR, NCOR2, MED12, RANBP2, ARID2, ZNF521, SETBP1, POLE, CTNNB1, EGFR, P53, and IDH. The tumor vaccine is preferably at least one selected from the group consisting of a P53 R273H mutant peptide, a KRAS G12V mutant peptide, and a KRAS G12C mutant peptide. The p53 R273H mutant peptide has an amino acid sequence preferably shown in SEQ ID NO: 7 (MGYQRIEDSSGNLLGRNSFEVHVCACPGRDRRTEEEN). The KRAS G12V mutant peptide has an amino acid sequence preferably shown in SEQ ID NO: 8 (MGYQRITEYKLVVVGAVGVGKSALTIQ).

The present disclosure further provides a preparation method of the allogeneic CAR-DC tumor vaccine, including the following steps:
constructing a recombinant lentiviral vector containing gene sequences encoding the CAR and the tumor vaccine;
conducting lentiviral packaging using the recombinant lentiviral vector to obtain a recombinant lentivirus; and
infecting the alloDC or a precursor cell or a progenitor cell thereof with the recombinant lentivirus to obtain or culture the allogeneic CAR-DC tumor vaccine.

In the present disclosure, a recombinant lentiviral vector containing gene sequences encoding the CAR and the tumor vaccine is constructed. The recombinant lentiviral vector preferably includes an expression element for expressing the gene sequences encoding the CAR and the tumor vaccine, the expression element for expressing the gene sequences encoding the CAR and the tumor vaccine includes a promoter, a CAR-encoding gene, an expression regulation element, and a tumor vaccine-encoding gene. The promoter preferably includes EF1A; and the EF1A has a nucleotide sequence preferably shown in SEQ ID NO: 4; the CAR-encoding gene has a nucleotide sequence preferably shown in SEQ ID NO: 1 (atggccttaccagtgaccgccttgctcctgccgctggccttgctgctccacgccgccaggccgcaggtgcagctgttggagtctgggggaggct tggtacagcctggggggtccctgagactctcctgtgcagcctctggattcacctttagcagctataccatgtcttgggtgcgacaggcccctggac aagcgcttgagtggatgggaaccattagtagtcgtggtacttacacctactatccagacagtgtgaagggccgattcaccatctccagagacaacg ccaagaactcactgtatctgcaaatgaacagcctgagagccgaggacacggctgtgtattactgtgcgagagaagctatctttactcactggggcc gtggcaccctggtcaccgtctcctcaggtggtggtggttctggcggcggcggctccggtggtggtggttctgacatccagttgacccagtctccat cctccctgtctgcatctgtaggagacagagtcaccatcacttgcaaggcgagtcaggacattaataactatcacagctggtaccagcagaaacctg gccaggctcccaggctcctcatctatcgtgcaaacagattggtagatggggtcccagacaggttcagtggcagcgggtatggaacagattttacc ctcacaattaataacatagaatctgaggatgctgcatattacttctgtctgaaatataatgtgtttccgtacacgttcggccaagggaccaaggtggag atcaaaaccacgacgccagcgccgcgaccaccaacaccggegcccaccatcgcgtcgcagcccctgtccctgcgcccagaggcgtgccggc cagcggcggggggcgcagtgcacacgagggggctggacttcgcctgtgatatctacatctgggcgcccttggccgggacttgtggggtccttct cctgtcactggttatcaccctttactgccgctggcctccttctgcagcttgttcgggaaaagagtcagttgttgctataaggaccaatagccaatctga cttccacttacaaacttatggagatgaagatttgaatgaattagatcctcattatgaaatgcgactgaagatccaagtgcgaaaggcagctataacca gctatgagaaatcagatggtgtttacacgggcctgagcaccaggaaccaggagacttacgagactctgaagcatgagaaaccaccacagtaa). The tumor vaccine-encoding gene has a nucleotide sequence preferably shown in SEQ ID NO: 2 (atgggctaccagaggatcactgaatataaacttgtggtagttggagctgttggcgtaggcaagagtgccttgacgatacagtaa) or SEQ ID NO: 3 (atgggctaccagaggatcgaagactccagtggtaatctactgggacggaacagctttgaggtgcatgtttgtgcctgtcctgggagagaccggc gcacagaggaagagaat). The expression regulation element preferably includes IRES; and the IRES has a nucleotide sequence preferably shown in SEQ ID NO: 5 (cccctctccctcccccccccctaacgttactggccgaagccgcttggaataaggccggtgtgcgtttgtctatatgttattttccaccatattgccgtc ttttggcaatgtgagggcccggaaacctggccctgtcttcttgacgagcattcctaggggtctttcccctctcgccaaaggaatgcaaggtctgttga atgtcgtgaaggaagcagttcctctggaagcttcttgaagacaaacaacgtctgtagcgaccctttgcaggcageggaaccccccacctggcgac aggtgcctctgcggccaaaagccacgtgtataagatacacctgcaaaggcggcacaaccccagtgccacgttgtgagttggatagttgtggaaa gagtcaaatggctctcctcaagcgtattcaacaaggggctgaaggatgcccagaaggtaccccattgtatgggatctgatctggggcctcggtgc acatgctttacatgtgtttagtcgaggttaaaaaaacgtctaggccccccgaaccacggggacgtggttttcctttgaaaaacacgatgataa). There is no particular limitation on a method for constructing the recombinant lentiviral vector, and any method for constructing a recombinant lentiviral vector known in the art, such as patent CN114457117A, can be used. There is no particular limitation on a packaging method of the lentivirus, and any packaging method of a lentivirus known in the art may be used, such as Example 1 in patent CN114457117A.

In the present disclosure, the alloDC or the precursor cell or the progenitor cell thereof is preferably derived from at least one selected from the group consisting of a PBC, a BMC, an HSC, an ESC, and an iPSC. When an infected cell is the precursor cell or progenitor cell of the alloDC, a DC can be prepared through differentiation culture.

The present disclosure further provides an anti-tumor pharmaceutical composition, including the allogeneic CAR-DC tumor vaccine or an allogeneic CAR-DC tumor vaccine prepared by the preparation method and a medically acceptable auxiliary material.

The present disclosure further provides use of the allogeneic CAR-DC tumor vaccine or an allogeneic CAR-DC tumor vaccine prepared by the preparation method in preparation of a drug for treating a cancer in a subject in need thereof.

In the present disclosure, the cancer is preferably a solid cancer being any one selected from the group consisting of breast cancer, colorectal cancer, pancreatic cancer, liver cancer, lung cancer, and ovarian cancer. There is no particular limitation on a method for preparing the drug, and any method for preparing a drug known in the art may be used.

The alloDC tumor vaccine, and the preparation method and the use thereof provided by the present disclosure will be described in detail below with reference to the examples, but they should not be construed as limiting the claimed scope of the present disclosure.

### Example 1

### Method for Constructing Humanized Mice

Immunodeficient mice (purchased from GemPharmatech, NCG, T001475) were irradiated with a sublethal dose, and humanized thymus tissue of approximately 1 mm³ was transplanted into a renal membrane of the immunodeficient mice. The wound was sutured after the surgery, and CD34⁺ HSCs were injected through tail vein after the mice woke up. Blood samples were collected 10 weeks after the surgery to detect the reconstruction of an immune system in the mice.

2 to 3 drops of venous blood were collected from the hind legs of mice, and the cells were centrifuged in EDTA-PBS buffer. Red blood cells were lysed with 1×ACK lysis buffer until the red blood cells were completely lysed and the solution became transparent. A supernatant was removed after centrifugation, and the cells were washed once with DPBS, and then anti-CAR antibody and anti-CD11c antibody were added to allow incubation, then stained, washed once, and resuspended in DPBS to allow flow cytometry analysis to confirm the successful reconstruction of humanized mice.

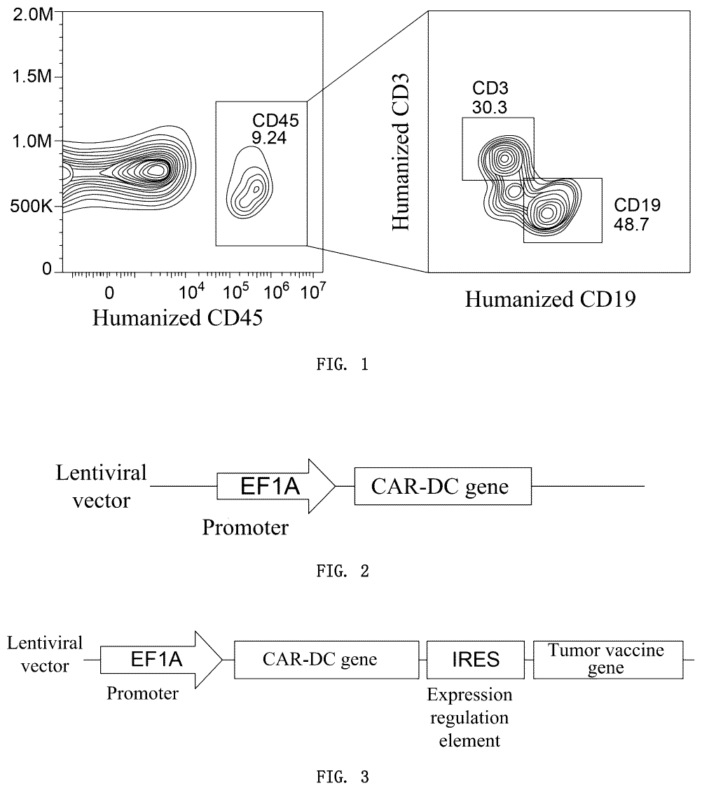

FIG. 1 showed a composition ratio of recombinant human CD45⁺ cells, CD3⁺ (T) cells, and CD19⁺ (B) cells detected by flow cytometry in peripheral blood taken after humanized mouse modeling.

### Comparative Example 1

### Preparation Method of Allogeneic CAR-DC

### 1. Construction of Recombinant Lentivirus Carrying CAR Gene

The construction of the recombinant lentiviral vector was to clone an expression element consisting of a promoter and a CAR gene into a lentiviral vector, where the CAR gene had a sequence shown in SEQ ID NO: 1; the promoter was EF1A sequence (SEQ ID NO: 4, gggcagagcgcacatcgcccacagtccccgagaagttggggggaggggtcggcaattgatccggtgcctagagaaggtggcgcggggtaaa ctgggaaagtgatgtcgtgtactggctccgcctttttcccgaggggggggagaaccgtatataagtgcagtagtcgccgtgaacgttctttttcgca acgggtttgccgccagaacacag); A schematic diagram of the expression element in the recombinant lentiviral vector was shown in FIG. 2. The construction of lentiviral vector and the preparation method of recombinant lentivirus referred to the description of Example 1 in patent CN114457117A. 2. Construction of CAR-tumor vaccine cells

The tibia of the successfully constructed humanized mouse was removed. BMCs were flushed down with a syringe. The flushed cells were repeatedly blown several times to disperse the bone marrow; and the bone marrow was further formed into a single cell suspension with a cell screen. The red blood cells were completely lysed with ACK lysis buffer, a supernatant was removed after centrifugation, and the cells were counted after washing with PBS. The obtained precursor cells were transferred to a 6-well plate to allow differentiation culture at a density of (5-10)×10⁵ per well/mL to obtain DCs, where a differentiation medium was RPMI1640 complete medium containing 20 ng/ml GM-CSF and 5 ng/ml IL-4.

The recombinant lentivirus carrying the CAR gene and the tumor vaccine gene was transduced into the DCs at an MOI of about 100, the lentivirus was incubated in a static state for 12 h. the differentiation medium was added to allow transduction for 24 h. the cells were collected, the virus was washed off, and the cells were further cultured in a fresh differentiation medium for later use. Detection of CAR receptors in DCs: the cells were stained with protein L-biotin, incubated at room temperature for 30 min, washed once with DPBS, incubated with PE Streptavidin at room temperature in the dark for 30 min. washed once, and transferred into 400 μL of DPBS to allow subsequent flow cytometry analysis.

### Comparative Example 2

### Preparation Method of Allogeneic CAR-DC Tumor Vaccine

1. Construction of recombinant lentivirus carrying CAR gene and tumor vaccine gene

The construction of the recombinant lentiviral vector was to clone an expression element consisting of a promoter, a CAR gene, an expression regulation element, and a tumor vaccine gene into a lentiviral vector, where the CAR gene had a sequence shown in SEQ ID NO: 1; the tumor vaccine gene was a gene sequence encoding Kras G12V tumor vaccine (SEQ ID NO: 2) or a gene sequence encoding P53 R273H tumor vaccine (SEQ ID NO: 3); the promoter was an EF1A sequence (SEQ ID NO: 4); the expression regulation element was an IRES sequence (SEQ ID NO: 5); the schematic diagram of the expression element in the recombinant lentiviral vector was shown in FIG. 3. The construction of lentiviral vector and the preparation method of recombinant lentivirus referred to the description of Example 1 in patent CN114457117A.

### 2. Construction of CAR-DC Tumor Vaccine

The tibia of the successfully constructed humanized mouse was removed. BMCs were flushed down with a syringe. The flushed cells were repeatedly blown several times to disperse the bone marrow; and the bone marrow was further formed into a single cell suspension with a cell screen. The red blood cells were completely lysed with ACK lysis buffer, a supernatant was removed after centrifugation, and the cells were counted after washing with PBS. The obtained precursor cells were transferred to a 6-well plate to allow differentiation culture at a density of (5-10)×10⁵ per well/mL to obtain DCs, where a differentiation medium was RPMI1640 complete medium containing 20 ng/mL GM-CSF and 5 ng/ml IL-4.

The recombinant lentivirus carrying the CAR gene and the tumor vaccine gene was transduced into the DCs at an MOI of about 100, the lentivirus was incubated in a static state for 12 h. the differentiation medium was added to allow transduction for 24 h. the cells were collected, the virus was washed off, and the cells were further cultured in a fresh differentiation medium for later use. Detection of CAR receptors in DCs: the cells were stained with protein L-biotin, incubated at room temperature for 30 min, washed once with DPBS, incubated with PE Streptavidin at room temperature in the dark for 30 min. washed once, and transferred into 400 μL of DPBS to allow subsequent flow cytometry analysis.

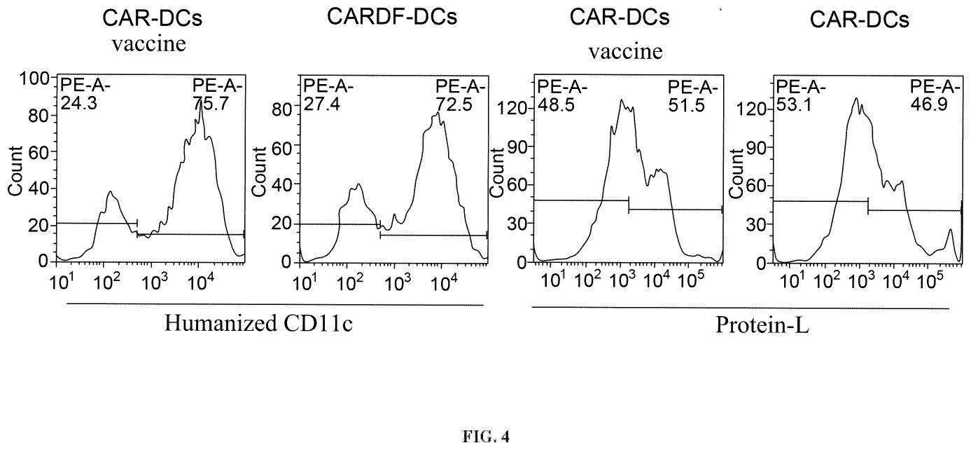

The flow cytometry analysis results of the CAR-DC constructed in Comparative Example 1 and the CAR-DC tumor vaccine constructed in Example 2 were shown in FIG. 4. The results showed that autologous BMCs differentiated into humanized DCs and then expressed CAR protein and CD11c markers on their surface.

### Example 2

### Preparation Method of Allogeneic CAR-DC Tumor Vaccine

Allogeneic DCs were derived from CD14 cells isolated from PBMCs in the peripheral blood of healthy individuals. Specifically. PBMCs were counted, incubated with CD14 magnetic beads at 4° C. for 15 min, washed once with MACS buffer (EDTA-PBS. 2% FBS), and the cells were passed through a pre-washed magnetic column. The magnetic column was rinsed with buffer 3 times. 3 mL each time. After the rinsing was completed, the magnetic column was removed from the magnetic stand, and the cells were washed down with 5 mL of buffer and collected. The cells were adjusted to (5-10)×10⁵ cells/mL, and a differentiation medium was: RPMI1640 complete medium supplemented with 100 ng/ml GM-CSF and 100 ng/ml IL-4. The two recombinant lentiviruses constructed in Comparative Example 2 were transduced into DCs at an MOI of about 100, the lentivirus was incubated in a static state for 12 h. the differentiation medium was added to allow transduction for 24 h. the cells were collected, the virus was washed off, and the cells were further cultured in a fresh differentiation medium for later use. Detection of CAR receptors in DCs: the cells were stained with protein L-biotin, incubated at room temperature for 30 min, washed once with DPBS, incubated with PE Streptavidin at room temperature in the dark for 30 min, washed once, and then transferred into 400 μL of DPBS to allow subsequent flow cytometry analysis.

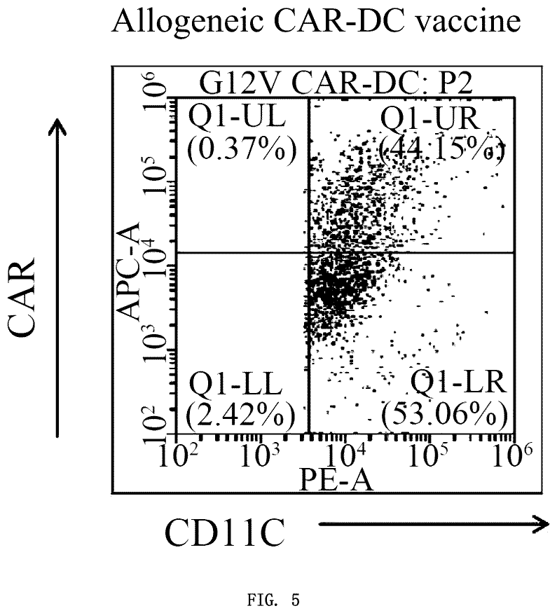

The results were shown in FIG. 5. PBMC-derived allogeneic monocytes were differentiated into humanized DCs and then expressed CAR protein on their surface.

### Example 3

### In Vivo Therapeutic Experiments of Autologous DC Tumor Vaccine and Allogeneic DC Tumor Vaccine

After the humanized mice were successfully prepared, the hair on the back of the mice was shaved, 1×10⁶ cells were resuspended in PBS, 50% matrigel was added, and the cells were transplanted subcutaneously into the mice using a 1 mL syringe. The tumor-bearing mice were treated after tumors were formed. The mice were divided into 4 groups: an untreated group, an autologous CAR-DC group, an autologous CAR-DC vaccine group (including CA-RDC-Kras^{G12V} and CAR-DC-P53^{R273H}), and an allogeneic CAR-DC vaccine group (including CAR-DC-Kras^{G12V} and CAR-DC-P53^{R273H}). The corresponding cells in each group were resuspended in 500 μL of PBS, and the cells were injected back into the mice through tail vein. The mice were continuously observed, and a tumor size was measured 1 to 2 times a week. The tumor volume was calculated by the following formula I:

Tumor volume=(width²×length)+2, Formula I.

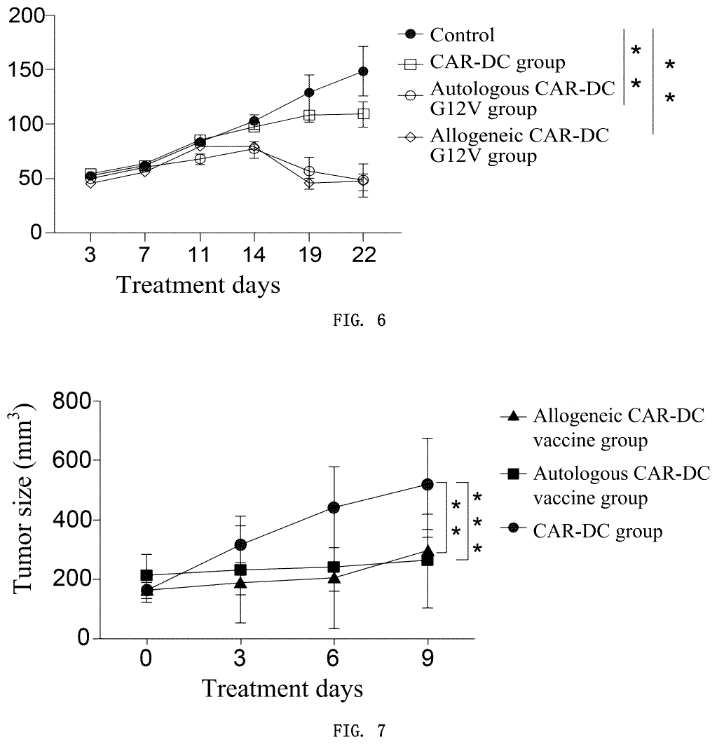

The results of tumor inhibition by allogeneic and autologous CAR-DC-Kras^{G12V} were shown in FIG. 6. The results showed that tumor growth was significantly inhibited after treatment with autologous CAR-DC vaccine and allogeneic CAR-DC vaccine, indicating that allogeneic CAR-DC vaccine cells could effectively inhibit tumors and even had the same therapeutic effect as autologous cells.

FIG. 7 shows tumor growth curves of each group of mice treated in vivo (CAR-DC-P53^{R273H}). FIG. 7 showed that tumor growth was significantly inhibited after treatment with autologous CAR-DC vaccine and allogeneic CAR-DC vaccine, indicating that allogeneic CAR-DC vaccine cells could effectively inhibit tumors and even had the same therapeutic effect as autologous cells.

The above are merely preferred implementations of the present application. It should be noted that a person of ordinary skill in the art may further make several improvements and modifications without departing from the principle of the present application, but such improvements and modifications should be deemed as falling within the protection scope of the present application.

What is claimed is:

1. An allogeneic chimeric antigen receptor (CAR) dendritic cell (DC) tumor vaccine, wherein the allogeneic CAR-DC tumor vaccine is an allogeneic dendritic cell (alloDC) that recombinantly expresses a CAR and a tumor vaccine.

2. The allogeneic CAR-DC tumor vaccine according to claim 1, wherein the alloDC is a cell therapy-targeted dendritic cell (DC) derived from different individuals of a same species.

3. The allogeneic CAR-DC tumor vaccine according to claim 1, wherein the CAR has an amino acid sequence shown in SEQ ID NO: 6.

4. The allogeneic CAR-DC tumor vaccine according to claim 1, wherein a source gene of the tumor vaccine is at least one selected from the group consisting of TP53, PIK3CA, LRP1B, KRAS, APC, FAT4, KMT2D, KMT2C, BRAF, ARID1A, FAT1, PTEN, ATM, ZFHX3, CREBBP, GRIN2A, NF1, PDE4DIP, PTPRT, TRRAP, RNF213, PREX2, SPEN, ERBB4, KMT2A, RB1, BRCA2, FBXW7, CARD11, PTPRB, ROS1, ATRX, UBR5, NOTCH1, POLQ, EP300, NCOR1, SETD2, SMARCA4, CHD4, MTOR, NCOR2, MED12, RANBP2, ARID2, ZNF521, SETBP1, POLE, CTNNB1, EGFR, P53, and IDH.

5. The allogeneic CAR-DC tumor vaccine according to claim 4, wherein the tumor vaccine is at least one selected from the group consisting of a P53 R273H mutant peptide, a KRAS G12V mutant peptide, and a KRAS G12C mutant peptide.

6. The allogeneic CAR-DC tumor vaccine according to claim 5, wherein the p53 R273H mutant peptide has an amino acid sequence shown in SEQ ID NO: 7.

7. The allogeneic CAR-DC tumor vaccine according to claim 5, wherein the KRAS G12V mutant peptide has an amino acid sequence shown in SEQ ID NO: 8.

8. A preparation method of the allogeneic CAR-DC tumor vaccine according to claim 1, comprising the following steps:
constructing a recombinant lentiviral vector containing gene sequences encoding the CAR and the tumor vaccine;
conducting lentiviral packaging using the recombinant lentiviral vector to obtain a recombinant lentivirus; and
infecting the alloDC or a precursor cell or a progenitor cell thereof with the recombinant lentivirus to obtain or culture the allogeneic CAR-DC tumor vaccine.

9. The preparation method according to claim 8, wherein the alloDC or the precursor cell or the progenitor cell thereof is derived from at least one selected from the group consisting of a peripheral blood cell (PBC), a bone marrow cell (BMC), a hematopoietic stem cell (HSC), an embryonic stem cell (ESC), and an induced pluripotent stem cell (iPSC).

10. The preparation method according to claim 8, wherein the recombinant lentiviral vector comprises an expression element for expressing the gene sequences encoding the CAR and the tumor vaccine; and
the expression element for expressing the gene sequences encoding the CAR and the tumor vaccine comprises a promoter, a CAR-encoding gene, an expression regulation element, and a tumor vaccine-encoding gene.

11. The preparation method according to claim 10, wherein the promoter comprises elongation factor 1 alpha (EF1A); and the EF1A has a nucleotide sequence shown in SEQ ID NO: 4.

12. The preparation method according to claim 10, wherein the CAR-encoding gene has a nucleotide sequence shown in SEQ ID NO: 1.

13. The preparation method according to claim 10, wherein the tumor vaccine-encoding gene has a nucleotide sequence shown in SEQ ID NO: 2 or SEQ ID NO: 3.

14. The preparation method according to claim 10, wherein the expression regulation element comprises internal ribosome entry site (IRES); and the IRES has a nucleotide sequence shown in SEQ ID NO: 5.

15. An anti-tumor pharmaceutical composition, comprising the allogeneic CAR-DC tumor vaccine according to claim 1 and a medically acceptable auxiliary material.

16. A treatment method of a cancer based on the allogeneic CAR-DC tumor vaccine according to claim 1, comprising injecting the allogeneic CAR-DC tumor vaccine into a cancer patient.

17. The treatment method according to claim 16, wherein the cancer is a solid cancer being any one selected from the group consisting of breast cancer, colorectal cancer, pancreatic cancer, liver cancer, lung cancer, and ovarian cancer.

18. The preparation method according to claim 8, wherein the alloDC is a cell therapy-targeted dendritic cell (DC) derived from different individuals of a same species.

19. The preparation method according to claim 8, wherein the CAR has an amino acid sequence shown in SEQ ID NO: 6.

20. The preparation method according to claim 8, wherein a source gene of the tumor vaccine is at least one selected from the group consisting of TP53, PIK3CA, LRP1B, KRAS, APC, FAT4, KMT2D, KMT2C, BRAF, ARID1A, FAT1, PTEN, ATM, ZFHX3, CREBBP, GRIN2A, NF1, PDE4DIP, PTPRT, TRRAP, RNF213, PREX2, SPEN, ERBB4, KMT2A, RB1, BRCA2, FBXW7, CARD11, PTPRB, ROS1, ATRX, UBR5, NOTCH1, POLQ, EP300, NCOR1, SETD2, SMARCA4, CHD4, MTOR, NCOR2, MED12, RANBP2, ARID2, ZNF521, SETBP1, POLE, CTNNB1, EGFR, P53, and IDH.


SEQUENCE LISTING (8 sequences)

SEQ ID NO: 1 - DNA, 1242 bp, synthetic construct
  source 1..1242: mol_type = other DNA; note = CAR-encoding gene
atggccttaccagtgaccgccttgctcctgccgctggccttgctgctccacgccgccaggccgcaggtgcagctgttggagtctgggggaggcttggtacagcctggggggtccctgagactctcctgtgcagcctctggattcacctttagcagctataccatgtcttgggtgcgacaggcccctggacaagcgcttgagtggatgggaaccattagtagtcgtggtacttacacctactatccagacagtgtgaagggccgattcaccatctccagagacaacgccaagaactcactgtatctgcaaatgaacagcctgagagccgaggacacggctgtgtattactgtgcgagagaagctatctttactcactggggccgtggcaccctggtcaccgtctcctcaggtggtggtggttctggcggcggcggctccggtggtggtggttctgacatccagttgacccagtctccatcctccctgtctgcatctgtaggagacagagtcaccatcacttgcaaggcgagtcaggacattaataactatcacagctggtaccagcagaaacctggccaggctcccaggctcctcatctatcgtgcaaacagattggtagatggggtcccagacaggttcagtggcagcgggtatggaacagattttaccctcacaattaataacatagaatctgaggatgctgcatattacttctgtctgaaatataatgtgtttccgtacacgttcggccaagggaccaaggtggagatcaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccgctggcctccttctgcagcttgttcgggaaaagagtcagttgttgctataaggaccaatagccaatctgacttccacttacaaacttatggagatgaagatttgaatgaattagatcctcattatgaaatgcgactgaagatccaagtgcgaaaggcagctataaccagctatgagaaatcagatggtgtttacacgggcctgagcaccaggaaccaggagacttacgagactctgaagcatgagaaaccaccacagtaa

SEQ ID NO: 2 - DNA, 84 bp, synthetic construct
  source 1..84: mol_type = other DNA; note = A gene sequence encoding Kras G12V tumor vaccine
atgggctaccagaggatcactgaatataaacttgtggtagttggagctgttggcgtaggcaagagtgccttgacgatacagtaa

SEQ ID NO: 3 - DNA, 111 bp, synthetic construct
  source 1..111: mol_type = other DNA; note = A gene sequence encoding P53 R273H tumor vaccine
atgggctaccagaggatcgaagactccagtggtaatctactgggacggaacagctttgaggtgcatgtttgtgcctgtcctgggagagaccggcgcacagaggaagagaat

SEQ ID NO: 4 - DNA, 212 bp, synthetic construct
  source 1..212: mol_type = other DNA; note = A nucleotide sequence of EF1A
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 5 - DNA, 574 bp, synthetic construct
  source 1..574: mol_type = other DNA; note = A nucleotide sequence of IRES
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 6 - protein, 413 aa, synthetic construct
  source 1..413: note = An amino acid sequence of CAR
MALPVTALLLPLALLLHAARPQVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTHWGRGTLVTVSSGGGGSGGGGSGGGGSDIQLTQSPSSLSASVGDRVTITCKASQDINNYHSWYQQKPGQAPRLLIYRANRLVDGVPDRFSGSGYGTDFTLTINNIESEDAAYYFCLKYNVFPYTFGQGTKVEIKTTTPAPRPPTPAPTIASQPLSLRPEACRPAAGGAVHTRGLDFACDIYIWAPLAGTCGVLLLSLVITLYCRWPPSAACSGKESVVAIRTNSQSDFHLQTYGDEDLNELDPHYEMRLKIQVRKAAITSYEKSDGVYTGLSTRNQETYETLKHEKPPQ

SEQ ID NO: 7 - protein, 37 aa, synthetic construct
  source 1..37: note = An amino acid sequence of p53 R273H mutant peptide
MGYQRIEDSSGNLLGRNSFEVHVCACPGRDRRTEEEN

SEQ ID NO: 8 - protein, 27 aa, synthetic construct
  source 1..27: note = An amino acid sequence of KRAS G12V mutant peptide
MGYQRITEYKLVVVGAVGVGKSALTIQ